(19) United States
(12) Patent Application Publication
(10) Pub. No.: US 2024/0180523 A1
(43) Pub. Date: Jun. 6, 2024

(54) A Portable Ultrasound Device and Method for Ultrasonic Imaging

(71) Applicant: Veintech Pty Ltd, Dianella (AU)

(72) Inventors: Nikhilesh BAPPOO, DIANELLA (AU); Nicholas BUCKLEY, DIANELLA (AU); Katherine ARENSON, DIANELLA (AU); Ritu MEHTA, DIANELLA (AU); Saleem Ahmed SALEEM AL ODEH, DIANELLA (AU)

(21) Appl. No.: 18/555,268

(22) PCT Filed: Apr. 19, 2022
(86) PCT No.: PCT/AU2022/050356

§ 371 Date: Oct. 13, 2023

(30) Foreign Application Priority Data
Apr. 19, 2021 (AU) 2021901145

(51) Int. Cl. A61B 8/00; A61B 8/06; A61B 8/08

(52) U.S. Cl. CPC A61B 8/4494; A61B 8/06; A61B 8/0891; A61B 8/4427; A61B 8/4472; A61B 8/488

(57) ABSTRACT

A portable ultrasound device (109) for non-invasively imaging a selected sub-cutaneous structure, comprising: (a) a housing (110); and (b) a plurality of arrays (300A) of transducer elements (300). Each array (300A) is arranged in parallel and each transducer element (300) comprises a transmitter transducer (705) and a receiver transducer (707), located within the housing (110) for continuously transmitting ultrasound energy in a predetermined frequency range toward a body (100) of a subject and continuously receiving echo signals in a predetermined frequency range from the subjects body (100). The plurality of parallel arrays (300A) enables imaging of the sub-cutaneous structure (101, 101A) in multiple transverse and lateral planes. The ultrasound device (109) further includes (c) a controller (250) for operating the plurality of arrays (300A) of transducer elements (300) and communicable with a processor (350) for processing received echo signals from the plurality of arrays (300A) of transducer elements (300); and (d) a screen (104) for displaying an interpretable image (104A, 104B) of said sub-cutaneous structure (101, 101A) produced by the processor (350).

### TECHNICAL FIELD

The present invention relates to a portable ultrasound device and method of ultrasonic imaging using the device.

### BACKGROUND ART

Peripheral intravenous cannulation (PIVC; placing a vascular access device, typically a plastic cannula, inside a vein with the help of an introducing needle) is the most commonly performed invasive medical procedure with over two billion cannulas sold worldwide each year (Rickard et al. (2018) Lancet 392(10145): 419-430). Over 70% of admitted patients will require a cannula during their admission (Zingg and Pittet (2009) Int J Antimicrob Agents 34 Suppl 4: S38-42).

The overwhelming majority of vascular access procedures using vascular access devices, such as cannulas, are performed without the aid of any visualisation device and rely on what is observed through the patient's skin and by the clinician's ability to feel the vessel. A patients' initial cannula is most commonly placed in the Emergency Department as this is the entry point for most admitted patients. These are often inserted in a rushed manner, involving guesswork in feeling or seeing veins, that results in a sub-optimal insertion location and method. Once the patient has stabilized, these PIVCs often need to be removed and reinserted.

Cannulas can be sited by doctors or nurses and this differs from country to country and hospital to hospital. In Western Australian public hospitals, the majority of cannulas are placed by junior doctors (Interns, Resident Medical Officers and Registrars) or trained clinical nurses.

When a cannulation is deemed difficult or there have been multiple failed attempts, junior doctors will enlist the help of senior doctors to reattempt the insertion. If multiple standard attempts fail then an appropriately trained, usually senior doctor, may use ultrasound to visualise a vein and guide further cannulation attempts. Ultrasound assisted cannulation or venepuncture requires significant training and experience and only a few departments have access to an ultrasound machine.

A major hurdle for poor ultrasound adoption is the difficulty in using the technology.

Firstly, end users require extensive training to adjust ultrasound settings and adequately position the hardware (probe) on the patient in order to obtain a clear image in the first place, let along interpreting the images.

Secondly, it is difficult for end users, particularly those that perform most cannulations (junior doctors or accredited nurses) to objectively interpret traditional B-mode or colour doppler images and to identify and discriminate between arteries or veins to target.

Thirdly, usability issues exist, including the screen typically being on a separate cart-based system and not aligned with the position of blood vessels, therefore requiring end users to observe an adjacent screen while holding an imaging probe and trying to insert a needle.

According to the literature, cannulation fails an unacceptable 40% of the time on first attempt (Rickard et al. (2018) Lancet 392(10145): 419-430; Cooke et al. (2018) PLoS One 13(2): e0193436; Keogh and Mathew (2019) Australian Commission on Safety and Quality in Health Care).

Multiple cannulation attempts are often required in patients with difficult venous access. Not only do these multiple attempts cause considerable pain and distress to the patients, but they also cause frustration to the clinicians and significant time and equipment wastage. This causes downstream delays to investigations and treatments. These delays also slow the flow of patients through the department increasing emergency wait times.

Repeated cannulation attempts are also associated with higher infection rates, with increased morbidity and longer length of stays. This leads to increased costs to the health service (Bahl et al. (2016) The American Journal of Emergency Medicine, 34(10): 1950-1954; Fields et al. (2013) Journal of Vascular Access 15(6): 514-518).

First pass cannulation attempts may fail in patients for a number of reasons. Some may be related to patient anxiety and consequent movement, more may be related to the lack of skill or experience of the clinician, however the majority are because a patient has difficult venous access, reducing vein identification visually or by feel. By way of non-limiting examples, this may be as a result of obesity, past or current chemotherapy, previous intravenous drug use or renal failure and cardiac failure.

Other patients that can be difficult to cannulate include, but are not limited to, the very young and the very old, as well as patients with dark skin (Au et al. (2012) Am J Emerg Med 30(9): 1950-1954; Bauman et al. (2009) Am J Emerg Med 27(2): 135-140; Brannam et al. (2004) Acad Emerg Med 11(12): 1361-1363; Chinnock et al. (2007) J Emerg Med 33(4): 401-405). Obesity is by far the most common cause for difficult vascular access, with 28% of Australians and 13% of people worldwide being obese (World Health Organisation (2018)).

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides for a portable ultrasound device for imaging a sub-cutaneous structure in a subject, for example, for assisting a practitioner in the performance of a venepuncture or cannulation.

In one aspect, the present invention provides a portable ultrasound device for non-invasively imaging a selected sub-cutaneous structure in a subject, comprising:
(a) a housing;
(b) a plurality of arrays of transducer elements, each array arranged in parallel and each transducer element comprising a transmitter transducer and a receiver transducer, located within said housing for continuously transmitting ultrasound energy in a predetermined frequency range toward a body of a subject and continuously receiving echo signals in a predetermined frequency range from the body of the subject following reflection of ultrasound energy, said plurality of parallel arrays of transducer elements enabling imaging of the sub-cutaneous structure in multiple transverse and lateral planes;
(c) a controller for operating said plurality of arrays of transducer elements and communicable with a processor for processing said received echo signals from said plurality of arrays of transducer elements; and
(d) a screen forming part of said housing for displaying said image of said sub-cutaneous structure wherein said processor is configured to process said echo signals returning from the sub-cutaneous structure to selectively produce an interpretable image of the sub-cutaneous structure of the subject on said screen.

It will be understood that the subject may be a human patient or a veterinary subject such as domestic animal, farm animal or laboratory animal

The portable ultrasound device is configured to facilitate operation by personnel without a specific specialisation in ultrasonic imaging. Typically, ultrasound operators have been required to manually adjust the probe's orientation, manually calibrate the operating field of view, gain levels, contrast levels, imaging depth or doppler parameters all while interpreting an abstract 2D grayscale image (or 2D colour-doppler) taken in a single scan line. The device is conveniently configured, as described below, or the controller of the portable ultrasound device is conveniently programmed to undertake one, more or all of these functions to avoid need for a user of the device to do so. For example, gain control may be made completely automatic without the user of the portable ultrasound device requiring to adjust gain. The portable ultrasound device may simply be provided with user functions that allow the device to be powered up and down and to select a favoured image mode, for example and preferably between B mode, colour Doppler mode and a schematic mode enabling display of a sub-cutaneous structure, for example a vascular structure. The user functions may, if desired, include a function to store an image as described below. In addition, in embodiments of the portable ultrasound device as described below, there is no need to manually adjust the probe orientation.

The sub-cutaneous structure is preferably a vascular structure, such as a vein or artery. However, the portable ultrasound device may be configured to alternatively, or additionally, enable imaging of other sub-cutaneous structures for nerve imaging and/or foreign body imaging.

Planar or linear arrays of transducer elements are desirable. The plurality of arrays of transducer elements may include any convenient number of arrays, say four arrays. Preferably, the plurality of arrays of transducer elements are spaced apart from each other by a distance ξ along the horizontal axis chosen to minimise interference and maximise scanning window. Most preferably, ξ is between 5 and 30 mm.

The arrays of transducer elements preferably comprise at least one Doppler transducer array. Preferably, the parallel transducer arrays are angled at an angle of insonation ϕ where 10<ϕ<60 to ensure the Doppler effect is captured and not nullified. Such an angled configuration also allows removal of need for an operator to manually adjust an operating field of view and imaging angles.

The plurality of parallel transducer arrays provides the ability to image the sub-cutaneous structure in multiple transverse and lateral planes to display its lateral position, up to a selected depth, for example 2-3 cm, below the skin surface in a contacting area where the ultrasound device contacts the skin of the subject. By providing simultaneous transverse and lateral imaging, images are effectively provided with a ‘3D’ appearance making the image easier to interpret, and use in effective cannulation, whether by personnel trained or untrained in ultrasonic imaging.

A linear array may be singular and affixed to a mobile track apparatus that allows the transducer array to move up the length of the vessel while being used to sense multiple ultrasound images for processing. Movement of the array on a track allows for imaging to occur in multiple planes, allowing for the combined lateral and transverse imaging mentioned previously. For example, rather than having say 4 crystal arrays in a fixed position, a single moveable array can achieve the same multiplanar function

A linear transducer may be single and oscillate around a fixed point whilst sensing multiple ultrasound images for processing.

The controller of the ultrasound device controls operation of the plurality of arrays of transducer elements to selectively transmit ultrasound energy to locate the sub-cutaneous structure which is then displayed on the screen following processing of the received echo signals by the processor. Where vascular structures are concerned, and vascular structures such as veins are described below for purposes of exemplification, candidate sub-cutaneous vascular structures are conveniently located in both transverse and lateral directions. This may involve transmitting ultrasound energy to the body of the subject to a selected depth of from less than 1 cm, with a dead zone about 5 mm, up to several centimetres, for example up to about 5 cm for a relatively narrow frequency range of 3-8 MHz, at which a candidate vascular structure is located. Transmission of ultrasound energy to greater depth is prevented to avoid receiving echo signals not reflective of a vascular structure and not of assistance in preparation for a cannulation procedure with a vascular access device such as a cannula or needle. This may involve, or also involve, processing echo signals to remove noise signals not reflective of candidate vascular structures. The processor enables updated display on the screen of the path of the venous structure as the portable ultrasound device is moved over the skin surface. The image quality is suitable for locating a suitable cannulation point to assist venepuncture

In preferred embodiments, the one-dimensional position (as an x-axis coordinate) of veins and arteries are identified across a singular transducer through a number of possible methods described below. This process advantageously occurs in real time for the plurality of transducers in the array and x-axis positions for each vein or artery locations can be stored and can be interpolated between transducers to create a venous or arterial path.

Advantageously, where sub-cutaneous vascular structures are of interest, the processor is programmed with instructions to discriminate between arterial and venous vascular structures. In an embodiment, a first algorithm allows such discrimination with an option being a time-domain based algorithm based on measurement of pulsatility. Veins are typically far less pulsatile than arteries, if pulsatile at all. Pulsatility metrics suitable for measurement and input to such an algorithm may include pulsatility index (i.e. (systole-diastole)/mean blood velocity in vessel), dicrotic notch, velocity reflection index (VRI), viscosity elastic index (VEI) and/or resistance index.

In another embodiment, the processor may be programmed with instructions to identify and discriminate between arterial and venous structures using an energy signal or energy signature to determine the position of a vein below the contacting area of the ultrasound device. The energy signal is preferably determined from a frequency-domain representation of the raw signal, determined by a Fast Fourier Transform (FFT) of the continuously acquired current signal or more preferably from a power spectral density (PSD) computed from the sampled signal. This energy signature is preferably the amplitude of the dominant signal frequency, or alternatively the sum of amplitudes of dominant frequencies or else the sum of amplitudes squared. More preferably the energy is the area under the FFT or PSD distribution. Alternatively, the energy could be the ratio of amplitude to area under the distribution or the ratio of sum of amplitudes (or sum of amplitudes squared) to the area under the distribution.

The processor may also be programmed with instructions to discriminate between veins and arteries due to intrinsic differences in the Doppler shift measured. If blood flow in veins is towards the transducer while the opposite occurs for arteries, the frequency shift can be either positive (veins) or negative (arteries). The converse is true if vein flow is away from the transducer and artery flow is towards the transducer. According to the directionality inferred from the Doppler Shift, the processor may determine a pseudo-negative magnitude for arteries, which also typically has a larger absolute magnitude than that of the vein. As such, veins and arteries can be discriminated.

The processor may also determine depth of the sub-cutaneous structure below the skin. For example, in the case of vascular structures, for a given vessel diameter, which may be calculated by the processor using a method such as those described below, characteristic curves may be inferred relating magnitude of energy to the vessel depth for different vessel diameters. This depth can be visualised numerically as a value in millimetres or centimetres below the skin, or through colour weightings by depth magnitude or through other visual representations such as 3D perception of shallowness or depth, all of which may conveniently be displayed on the screen. Thus, the processor may calculate depth without the need for B-mode cross sectional image reconstructions.

Alternatively, the processor may be programmed within instructions to perform B-mode imaging for each transducer element. Structural metrics (such as diameter or dimension, compressibility, and other curve features) can be calculated and used to discriminate between arteries and veins as described below. Machine learning techniques, for example including the use of convolutional neural networks, can be used to classify and identify veins and arteries, following training datasets provided by a population of ultrasound users. In B-mode or colour Doppler representations, depth of vessels can be directly measured and displayed on the screen of the ultrasound device.

In another embodiment of an algorithm for artery or vein discrimination, B-mode and Colour Doppler images are acquired and automatic computer vision techniques may be used to identify flow away or towards the transducer (or flow away from the heart, representing arteries, and flow towards the heart, representing veins). Colour Doppler represents flow towards the transducer (veins typically) as shades of blue with intensities representing velocity magnitudes, and flow away from the transducer (arteries typically) as shades of red. As such, arteries or veins can be distinguished through automatic computer vision techniques discriminating colour. In B-mode or colour Doppler representations, depth of vessels can be directly measured and displayed.

Conveniently, where energy signals are captured, the algorithm—as embodied in instructions programmed in the processor—recognizes that the peak of the energy signal or energy signature, forms part of parabolic or gaussian resembling distributions and that the start and end points of such distributions can be used to estimate structure sub-cutaneous dimension or diameter, particularly in the lateral direction.

Ultrasound data may be acquired by the processor for processing without and with mechanical compression of the subcutaneous vessels through application of external force by the housing of the ultrasound device onto the skin. Taking advantage of different vascular wall properties of veins (compressible but not muscular and not elastic) and arteries (muscular and elastic), changes during compression can be measured to distinguish between arteries and veins.

In one embodiment of the compression method, peak energy signals (determined from either FFT or PSD) change with compression. Notably, the positive peak (representing a flow in a vein when flowing towards the transducer) is flattened with compression while the negative peak (representing a flow in an artery when flowing away from the transducer), only shows reductions in magnitude while maintaining its directionality (negative peak representing flow away from the transducer). The characteristic drop in the peak (toward zero) for the vein and a linear decrease in magnitude for the artery are preferably calculated through absolute value measurements or more preferably through feature curve analysis to discriminate between arteries and veins. Alternatively, as vessel diameter can be determined from energy curves as previously described, the change in size due to compression can be estimated by calculating the change in diameter from before compression to after compression. A large deformation (say equivalent to 90-100% of the original diameter) is expected for veins compared to arteries. Preferably, the method is applicable to sub-cutaneous structures, i.e. veins or arteries, up to 5 cm in depth but most preferably up to 2-3 cm in depth. Vessel elasticity may also be determined.

In another embodiment of the compression method utilizing B-mode or colour doppler, automatic feature edge recognition is used to identify circular or elliptical objects and the maximum vertical chord length of said objects can be measured. Circular or elliptical objects can be identified as veins or arteries as the reduction in maximum vertical chord length is largest for veins (say equivalent to 90-100% of the original diameter) compared to arteries.

In another embodiment of both compression methods described, the compressibility of the vein either through energy drops or chord length drops can be used to indicate the structural integrity of the vein with larger resistance to compression indicating higher stiffness (structural integrity). Additionally, the restoration of structure following compression can be used to determine the extent of plastic deformations and therefore can be used to determine elasticity. The choice of structurally stable veins through both stiffness and elasticity inferences is a critical factor for cannula insertions as well as cannula success throughout indwelling periods (no dislodgement, extravasation).

In another embodiment, where both diameter and path of a vascular structure is available to the processor, the processor may be programmed with instructions to create a three dimensional structure of that vascular structure. The path data is used as a centreline and diameter data for each point may be used to create idealized circular cross sections with constant diameter for the energy based algorithm approach) or subject specific vascular cross sections which may or may not be circular (for B-mode or colour doppler approach). These cross sections can then be lofted along the vessel path to create three dimensional structures, with triangulated surfaces created by algorithms such as marching cubes.

The processor desirably includes a graphics processor which assists in providing requisite image quality in terms of providing sufficient information to identify the candidate vascular structure without reduction to a simple schematic which provides too little information to assist with cannulation.

Processing of received echo signals during ultrasound investigation involves numerous complex calculations which typically makes medical ultrasound equipment non-portable due to the wide range of ultrasound investigations that such equipment is called upon to perform. With the present procedure restricted, in preferred embodiments, to locating candidate vascular structures for cannulation, this complexity reduces but is still present. To that end, the processor—though it is desirably accommodated within the housing—may involve processing units located outside the housing but communicable through wire or wireless network with the portable ultrasound device to enable display of a candidate vascular structure on the screen forming part of the housing of the portable ultrasound device.

Processing involving processing units located outside the housing, if necessary because inclusion of the processor within the housing of the device is most preferred, may be performed using a cloud-based system such as Amazon AWS, whereby data are wirelessly transmitted, processed and returned to the device. Preferably, trivial and junk signals are automatically excluded while Doppler frequencies are calculated for non-trivial signals and stored on the temporary sampling memory. Preferably, the temporary sampling memory will be less than 8 GB, more preferably less than 6 GB or most preferably less than 4 GB or 2 GB. Wireless communications from portable ultrasound device to processing units or storage devices may be made, for example, by means of reversible USB-C, Bluetooth 5.0, or wireless internet in the form of standard WiFi, dual-band, Wi-Fi Direct or hotspot. In remote usage, in the absence of wireless internet, in one embodiment, the portable ultrasound device may have standard GSM or CDMA or HSPA or EVDO or LTE network technology paired with single SIM (nano SIM).

The housing of the portable ultrasound device is conveniently provided with a transducer array base. In this embodiment, the base of the housing may comprise the plurality of arrays of transducer elements padded with a backing layer and a matching layer. A transducer array base, or the base of the housing otherwise, may include at least one sensor for example selected from the group consisting of temperature sensors, heart rate sensors, cardiovascular sound sensors, bowel sound sensors, sweat analyte sensors, skin stiffness sensors and skin microbial sensors.

Advantageously, the plurality of arrays of transducer elements are arranged, first, to direct ultrasound energy in a predetermined frequency range at an angle of insonation m to a subcutaneous region of interest of the subject's body, preferably as instructed by the controller including, or communicable with, processing blocks for the analog front end (AFE), beam former with front end processing, and the backend processing block. The AFE may be implemented in the form of a fully integrated chip single-chip per 2, 4, 8, 16, 32 etc channels or in a multichip per channel solution. The AFE block may be implemented through field programmable gate array (FPGA) or ASIC advantageously implemented using chip(s). An FPGA based controller, which may store and generate pre-programmed digital signals containing ultrasonic oscillation settings is an option.

The beamformer comprises two parts, the transmit beamformer that has the function of initiating scan lines and generating a timed digital pulse string to the transducer elements. The digital pulse string is internally converted into high voltage pulses for the transducer elements so that the transducer elements transmit ultrasound energy in the predetermined frequency range. The receive beamformer has the function of receiving the echo signals from the AFE in the predetermined frequency range and collating the data into representative scan lines through filtering, windowing, summing and demodulation. The two beamformers are time synchronised and continuously communicate timing, position and control data to each other. The receive beamformer is also preferably implemented by FPGA for the portable ultrasound device.

The back-end processing block preferably includes B-mode, Doppler (preferably pulse wave doppler or most preferably continuous wave doppler) and colour flow processing functions and a user of the portable ultrasound device may toggle between these. The B-mode receives demodulated and compressed scan lines and uses interpolation and gray scale mapping to form 2-D gray scale images from the scan lines produced by the receive beamformer. The backend processing block, in this case, preferably produces an image that is ready for use by non-specialist staff in conducting a cannulation. This may involve use of an enhancement technique such as frame smoothing and/or edge detection.

Desirably, the screen forming part of the portable ultrasound device is a colour display unit with non-limiting examples being LCD, LED, OLED, AMOLED or trans-reflective with or without memory in pixel displays. The screen is preferably compatible with touchscreen functionality and is preferably flat. The sub-cutaneous, preferably vascular, structure is conveniently displayed on the screen (which comprises 1280×720 pixels, preferably less than 720×480 pixels, more preferably less than 640×360 pixels and most preferably less than 215×180 pixels) in real time and in a manner not requiring the user of the portable ultrasound device to interpret an ultrasound image alone. This differs from the range of operations conventionally performed by ultrasound operators such as manually adjusting an ultrasound probe's orientation, manually calibrating the operating field of view, setting gain levels, setting contrast levels, setting imaging depth or doppler parameters, all while interpreting an abstract 2D grayscale (or 2D colour-doppler) taken in a single scan line.

The screen, or display unit of which it forms part, has one or more of the following characteristics: preferably lightweight, preferably hydrophobic, preferably oleophobic and preferably chemically resistant to allow for easy sterilisation.

The screen desirably, conveniently with the assistance of the processor, provides an indication of the correct location for insertion of a vascular access device in the form of a cannula or like device. To that end, representation(s) on the screen may display information including one or more of: the depth of an imaged sub-cutaneous structure; and the position of a needle tip being inserted into the tissue. The representation(s) on the screen may indicate one or more of: the calibre of the imaged sub cutaneous structure, for example a vascular structure, and the position of a needle tip being inserted into the tissue. In embodiments of such processing and software indications, algorithms advantageously automatically recognize when a cannula is inserted into a vein and may represent that insertion for both a singular transducer or as the schematic representation of the path segment. For example, in an embodiment involving a schematic vein path representation determined from peak energy signatures (determined from either FFT or PSD), the original peak may become a trough or, alternatively, a “trough-like” shape such as a square or triangular dip in the energy signature, indicating disruption to the flow-derived energy as a result of the cannula tip disrupting the flow.

In another embodiment in which a B-mode or colour doppler method is used, image enhancement methods may be used to accentuate the intensity and acoustic reflection off the needle tip within both tissue or inside a vein and a bright spot may be represented to display the cannula tip at depth. In this embodiment, the location of the cannula tip within the vein can be used to create a schematic vein path representation, indicating the segment of the vein homing the cannula.

The processor may be programmed with instructions to calculate an optimal needle gauge and/or insertion angle recommended for access to the imaged sub-cutaneous structure, for example a vascular structure, by the vascular access device. The processor may be provided with instructions to select the vascular access device (cannula type) based on selected parameters which may, for example, include the group of vascular diameter, length of vein path and expected flow rates of both vein flow or infusion of fluids such as drugs.

As previously described, path of a vascular structure can be determined through interpolations between a plurality of transducer elements. In an embodiment involving a schematic vein path representation determined from peak energy signatures (determined from either FFT or PSD), vein diameter may be determined based on the features of the characteristic energy-position curves.

Automatic feature recognition methods can be used in embodiments of B-mode or Doppler ultrasound to automatically determine circular or elliptical objects within the field of view and vessel diameter or ellipticity can be calculated automatically by the processor. Based on vessel diameter or ellipticity, cannula size may be determined by the processor and displayed on the screen.

The processor can also be programmed with instructions to determine blood flow rates or velocities in a vascular structure using ultrasound data processed by the processor. Given fluid flow rates through cannulas need to ideally match venous blood flow rates, the processor is conveniently programmed with instructions to select and display a recommended cannula dependent on blood flow rate.

Representations in 3D may be provided by the processor and displayed on the screen for both vascular structure and haemodynamic fields such as, but not limited to one or more of: flow rate, velocity, pressure, shear stress, turbulence, stagnation, pulsatility or stenosis. Such measurements may be represented as time dependent, for example in the form of averaged graphs or real time display of the magnitude of the calculated haemodynamic parameter overlaid on reconstructed 3D structures. Calculated haemodynamic parameters such as shear stress or turbulence can be indicative of disruption of flow due to the inserted vascular access device, for example a cannula. Such parameters may be monitored to facilitate a cannulation process, for example—and without limitation—by determining a recommended time for replacement of the vascular access device and/or to assess the effectiveness of vein flushing with saline to keep the vein open and otherwise manage patency of the vein.

The portable ultrasound device may be used to determine a vascular structure, or vessel, most suited for selected drug infusions. Better flow in larger vessels, for example the cubital fossa of the forearm, is generally more suitable for iron or potassium infusions. Such vessels clear the infused treatments and tend to be less irritated and damaged compared to smaller, superficial vessels with much slower flows. The processor may process vessel structural characteristics and measured haemodynamic parameters to determine the optimal vessel for delivery of a selected infusion.

Mechanical indication for cannulation may also be provided where the portable ultrasound device housing is provided with a base plate with a needle guide to guide the placement of a cannula. The needle guide may include a notch, which is preferably a triangular or rectangular extrusion cut to the front of the base, but most preferably a spherical cut. Preferably, the notch will be less than 3 mm in size, and more preferably less than 1.5 mm in size, in order to restrict movement along the plane of needle insertion.

The screen, or display unit of which it forms part, may be hinged or flexed to move between a display position where it is at an acute angle to the housing and a position in which the screen may be flattened onto the housing of the device, making it possible to reduce the volume consumed while allowing for desirable user ergonomics during usage. This is particularly useful in paramedical, agricultural, military or other on-field applications of the technology where equipment space is constrained. The portable ultrasonic device may be configurable to allow display of the image on another device such as a computer or smartphone screen. However, this is not essential and there is no requirement to require a further device to display the image as a screen is provided as part of the housing of the present portable ultrasound device.

A base of the housing of the portable ultrasound device is conveniently placed in contact with the skin of a patient, the base thus having a contacting area with the skin of the patient. The base preferably has a curved shape, optionally being concave away from the skin surface, allowing convenient placement close to a limb, for example an upper limb such as the forearm of a human subject. The base of the portable ultrasound device may have a surface area of less than 150 cm², preferably less than 60 cm². More preferably, the surface area of the base will be less than 50, 45, 40 or 35 cm², most preferably less than 30 cm² or 25 cm².

The portable ultrasound device is also conveniently lightweight, the mass of the device preferably being no greater than 400 g, preferably less than 350 g, more preferably less than 200 g and most preferably less than 100 g or even 75 g.

Preferably, the portable ultrasound device includes a memory to store images and other data, such as patient and location data, if required. Preferably, an internal device storage is installed—conveniently as a microSDXC—of less than 512 GB capacity, more preferably less than 256 GB or 128 GB. When full, storage can be easily deleted or transferred to a reference back up drive through means described above. Alternatively, or additionally, to storage onboard the device, storage may be remote with communications between portable ultrasound device and storage device being implemented as described above. Data communicated in this way may be encrypted dependent on the application.

Data obtained from, or from using, the portable ultrasound device may be securely transmitted to a cloud-based, or other, system operated by an organisation utilising it. The system may store electronic medical records or information for insurance purposes. In one advantageous embodiment, the portable ultrasound device may be used in a pathology or blood collection application as results of blood tests can be automatically tracked and updated from the time of collection.

A handheld ultrasonic scanner, conveniently in the form of a cylindrical pipe, may be connected to the housing of the portable ultrasound device, for example through a USB port provided in the housing. The scanner desirably has Doppler functionality (preferably pulse wave doppler or most preferably continuous wave Doppler) and hence the ability to image sub-cutaneous structures, especially vascular structures, when the base of the device is too large for the surface of the skin of an imaged subject, such as—but not limited to—neonates, paediatrics or medium to small animals, including but not limited to dogs, cats, rodents, birds or rabbits. The scanner may also be useful when imaging is required in tight corners, creases or localised areas of physiological interest in the subject. Preferably, the scanner is less than 35 cm³in volume, more preferably less than 25 or 20 cm³in volume, most preferably less than 15 cm³in volume. Preferably, a skin contacting base of the scanner is less than 1 cm²in area, more preferably less than 0.75 cm² or 0.6 cm²in area and most preferably less than 0.5 cm²in area. Preferably, at least one of processing, storage and display of images is performed using the housing of the portable ultrasound device which serves as a base unit for the attached hand held scanner.

The portable ultrasound device may include a reading of a tracking device, such as a QR code or barcode. This enables scanning of tracking devices containing relevant data, for example standard electronic patient data including—but not limited to one or a plurality of—patient ID, age, sex, key patient history, location of ultrasound imaging, time of ultrasound imaging and reason for ultrasound imaging. Barcodes, for example, are typically used to store such data in hospitals, clinics, pathology or blood collection centres and other clinical or non-clinical settings.

Conveniently, the housing also includes a power source, such as a rechargeable battery for powering the portable ultrasound device. Connection to other power sources is also possible. The housing may include a DC to DC converter to boost battery voltage to the voltage, say up to 200 volts, to allow the controller to excite transducer elements to transmit ultrasound energy.

In another aspect, the present invention provides a method for imaging a sub cutaneous structure in a subject, comprising:
non-invasively transmitting ultrasound energy in a predetermined frequency range to the body of the subject via a plurality of arrays of transducer elements contained in a portable ultrasound device applied at or proximate to a location on the body of the subject, each array of said plurality of arrays of transducer elements being arranged in parallel and each transducer element comprising a transmitter transducer and a receiver transducer, said plurality of parallel arrays of transducer elements enabling imaging of the sub-cutaneous structure in multiple transverse and lateral planes;
receiving echo signals in a predetermined frequency range from the body of the subject following transmission of ultrasound energy;
processing said received echo signals with a processor; and
producing an image displaying the sub-cutaneous structure of the subject on a screen forming part of the portable ultrasound device.

The portable ultrasound device, as described above, is conveniently brought into contact with the subject and, more particularly a limb such as the forearm of a human patient. Conveniently the user deploys a detachable fastening means to secure the portable ultrasound device in position without needing to manually hold it in position. The detachable fastening means may be a single or multiple use strap, band or belt, preferably made of common medical grade material such as fabric or silicone. Preferably, the fabric material will be compatible with standard sterilization and cleaning mechanisms though may be disposable. A hypo-allergenic non-latex based material would be suitable for a disposable fastening means.

In another embodiment of the fastening affixation method, the strap can be in the form of a sleeve, wrap or cuff which attaches to the device and contacts a larger surface area of the limb.

The fastening means desirably allows adjustment of fastening force, for example through an adjustable clasp. The fastening force, similarly to a commonly used tourniquet, is advantageously selected to increase intra vascular pressure and hence engorge the vessel and improve visibility and reduce blood velocity to a range pre-programmed to be extracted by digital processing algorithms.

Following, positioning and optional fastening of the portable ultrasound device proximate the user is assisted to locate an optimised location of the vascular structure, typically a vein, for cannulation. As described above, a base plate of the portable ultrasound device base plate is conveniently provided with a needle guide to guide the placement of a cannula through alignment of the image of the selected vein with the needle guide. The needle guide may include a notch, in which case, alignment is made between the notch and the image of the selected vein. This feature facilitates selection of correct orientation for the portable ultrasound device without guesswork or excessive manual adjustment.

The user can then proceed to inserting a needle or cannula, being assisted by the needle guide and the image displayed on the screen, which act to guide the user to an optimal spot of cannula insertion.

A consumable acoustic conductive patch may be applied to the base of the portable ultrasound device prior to use. Preferably, a double-sided consumable acoustic conductive patch is attached to the base of the portable ultrasound device, conveniently through means of a medically compatible removable glue or a double-sided sticky tape. A sticky affixation end of the consumable patch conveniently attaches to the base of the device. A portion of the consumable patch may be peeled off to expose a medically sterile gel, contained within said patch, which aims to reduce conductance of ultrasonic waves through air, typically the cause of signal noise and signal loss, while maintaining the sterile field needed for venepuncture. Preferably, the volume of the sterile gel in such a preferred patch is less than 120 cm³. More preferably, the volume of the gel in such a preferred patch is less than 110 cm³ or 100 cm³ but most preferably less than 90 cm³.

A consumable conductive patch, preferably including an external sterile gel pocket, may conform with the form factor of the portable ultrasound device, for example being provided in the form of a custom sleeve, envelope or pocket. Such a patch may cover the portable ultrasound device, conveniently being applied thereto through a medically compatible removable glue. The external sterile gel pocket may conveniently be exposed through means of peeling as described above.

The portable ultrasound device and imaging method described above do not require any specialized needles or syringes for cannulation. It allows use of any conventional vascular access devices, such as catheters in any form.

The portable ultrasound device as described is self-contained, conveniently handheld, low cost and simply structured. In contrast to conventional ultrasound equipment, the portable ultrasound device of the present invention can readily be placed into correct orientation for determining a sub-cutaneous structure, for example for cannulation, and does not require complex practitioner adjustments or modular and separate system components consisting of scanning probes, processing units and monitors. The key components, including the transducer elements, processor and circuitry and screen are packaged in a single housing. The portable ultrasound device is conveniently lightweight and may be used to assist cannulation and other ultrasonic imaging of sub-cutaneous structures by a wider range of personnel without formal specialised training including medical and paramedical professionals such as, but not limited to, registered nurses, laboratory phlebotomists, therapists and researchers.

### BRIEF DESCRIPTION OF THE DRAWINGS

Further features of the portable ultrasound device and sub-cutaneous imaging method of the present invention are more fully described in the following description of preferred non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 shows a diagrammatic representation of a portable ultrasound device of one embodiment of the present invention and contacted with a forearm of a human patient.

FIG. 2 is a block diagram for the portable ultrasound device of FIG. 1.

FIG. 3a shows a side cross-sectional view of the housing of the portable ultrasound device of FIG. 1.

FIG. 3b shows top and side views of the transducer base and arrangement of transmitter and receiver crystal arrays forming transducer element arrays for the portable ultrasound device of FIG.

FIG. 4 is a flow diagram of one embodiment of the method for imaging a sub cutaneous structure of the present invention.

FIG. 5 shows a schematic representation of the transducer elements in a base of the portable ultrasound device of FIG. 1.

FIG. 6 shows the output of the method of FIG. 4 on the integrated display.

FIG. 7a is a schematic diagram of the base of the portable ultrasound device of FIG. 1 contacting a conductive material on the skin of a patient.

FIG. 7b is a schematic diagram of various components of a single transducer element shown in FIG. 7a involved in scanning a vessel.

FIG. 8 is a schematic diagram showing transformation of amplitude vs time signals to amplitude vs frequency data by Fast Fourier Transformation (FFT) according to an embodiment of the method of the invention.

FIG. 9 is a schematic diagram showing discrimination of arterial and venous structures through an energy signal based algorithm according to an embodiment of the method of the invention.

FIGS. 10(a) and (b) are schematic diagrams showing determination of vascular structure depth by an energy signal based algorithm according to an embodiment of the method of the invention.

FIG. 11 is a schematic diagram showing vascular structure characterisation through B mode imaging according to an embodiment of the method of the invention.

FIG. 12 is a schematic diagram showing vascular structure characterisation through B mode and colour doppler imaging according to an embodiment of the method of the invention.

FIG. 13 is a schematic diagram showing estimation of vascular diameter based on an energy signal according to an embodiment of the method of the invention.

FIG. 14 is a schematic diagram showing vascular structure characterisation from ultrasound imaging using a compression method according to an embodiment of the invention.

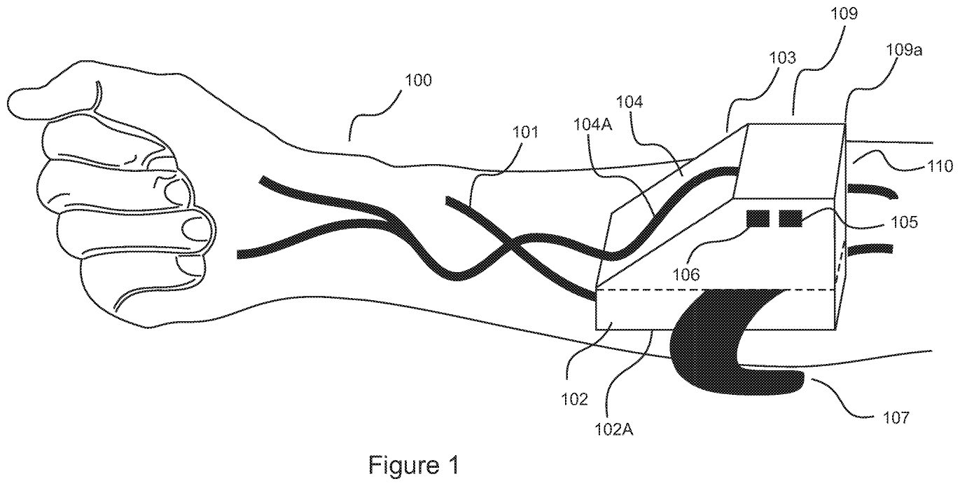

FIG. 1 shows a self-contained portable ultrasound device 109 that is used, through continuous transmission and receiving of ultrasound energy, to detect sub-cutaneous structures, here peripheral veins 101 at a depth of 2 to 3 cm below the skin of a patient's forearm 100 with the object of assisting cannulation. A sub-cutaneous structure could be at a lower depth, for example 5 mm, determined by the dead zone of the portable ultrasound device 109. The portable ultrasound device 109 is self-contained, hand-held, low-cost and simply structured with excessive circuitry required for whole body imaging not necessary for vascular imaging being excluded to reduce size and processing requirements. Portable ultrasound device 109 can be used by personnel without specialist knowledge of ultrasound imaging and who perform most cannulations (typically junior doctors and nurses).

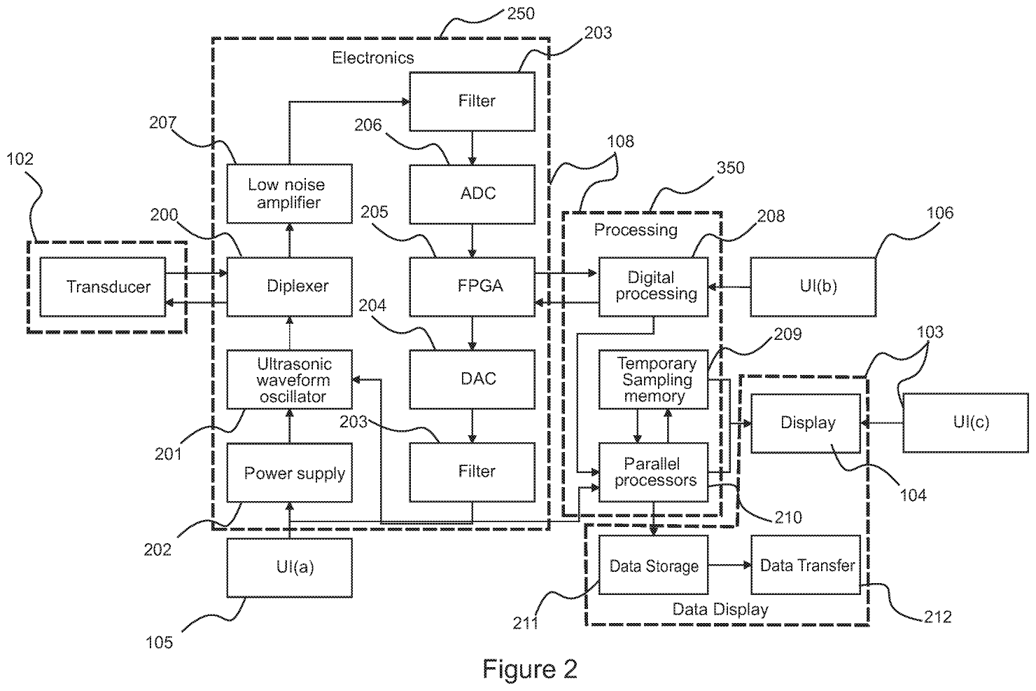

In contrast to conventional complex ultrasound equipment systems, such as those used for whole body imaging, portable ultrasound device 109 does not include multiple user controls or require modular and separate system components consisting of scanning probes, external processing units and monitors together with a requirement for fine adjustments of a number of ultrasound equipment parameters (such as gain) as would be understood for conventional ultrasound imaging equipment in the art of medical ultrasonic imaging; all key components including the ultrasonic transducer elements 102, controller 250 and processor 350 including processor blocks 208-210, power (FIG. 2: 202) and an integrated display unit 103, including a screen 104, are packaged on-board portable ultrasound device 109 in a single housing 110 which makes its use easier than current options.

Digital processing algorithms simplify traditional B-mode or colour doppler (preferably pulse wave doppler or most preferably continuous wave doppler), ultrasound images which can be abstract to interpret, to provide a two dimensional image of a candidate vein 101 for cannulation on screen 104 which, forming part of housing 108, is easier to observe than the adjacently disposed screens of prior practice. Rather than reconstructing raw signals (amplitude and frequencies) into grayscale images (B mode) which involves mapping corresponding signal receiving transducer crystal positions, the raw signals (and corresponding positions) are processed simply through frequency domain signal processing and discrimination methods, such as through pulsatility metrics as described above. Pulsatility index, being defined as (systole-diastole) divided by mean blood velocity in the vessel, may be used in this embodiment though other metrics are available as described below.

Frequency domain signal processing and discrimination methods represent less computation for the reconstruction of B mode images and less storage (both temporary and permanent) making hardware requirements simpler However, the user may choose to visualise and store raw images (B mode) of the subcutaneous structure, using the display mode switch 106 if preferred.

The integrated display unit 103, screen 104, power switch 105 and display mode switch 106 represent the exclusive and simple user interfaces for key functions for portable ultrasound device 109. Due to the configuration of the controller 250 and processor blocks 208-210 of processor 350 for the portable ultrasound device 109, there is no need for the user to access, additional functions (such as gain) or be provided with functionality to adjust the field of view, for gain adjustment or contrast adjustment. If adjustment of such settings is necessary, an authorised user—such as a maintenance engineer or repairer—could make such adjustments. This would not typically occur, or required to occur, during the cannulation procedure.

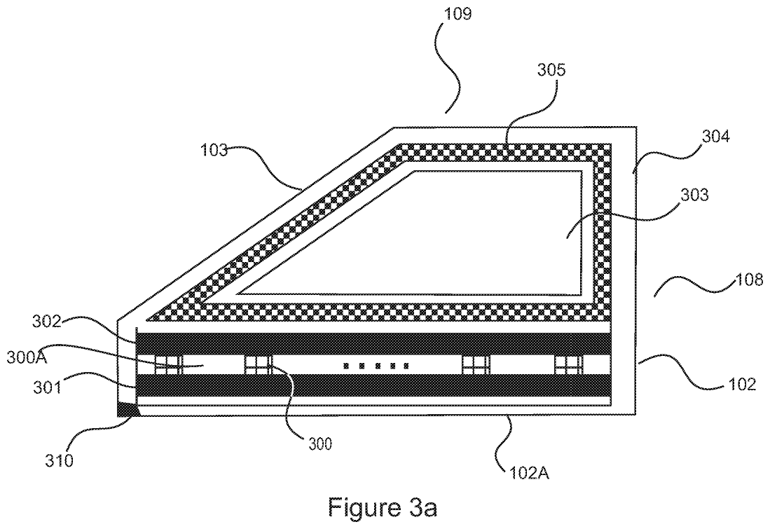

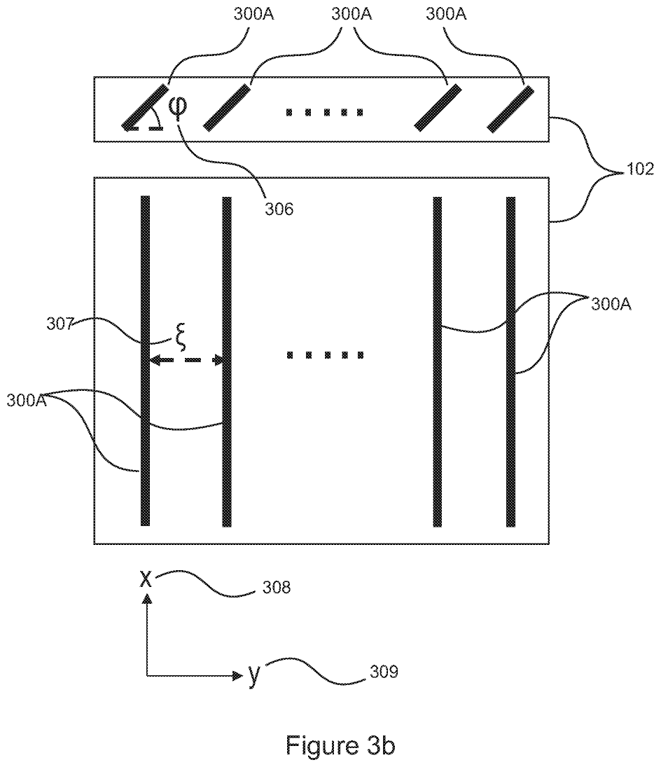

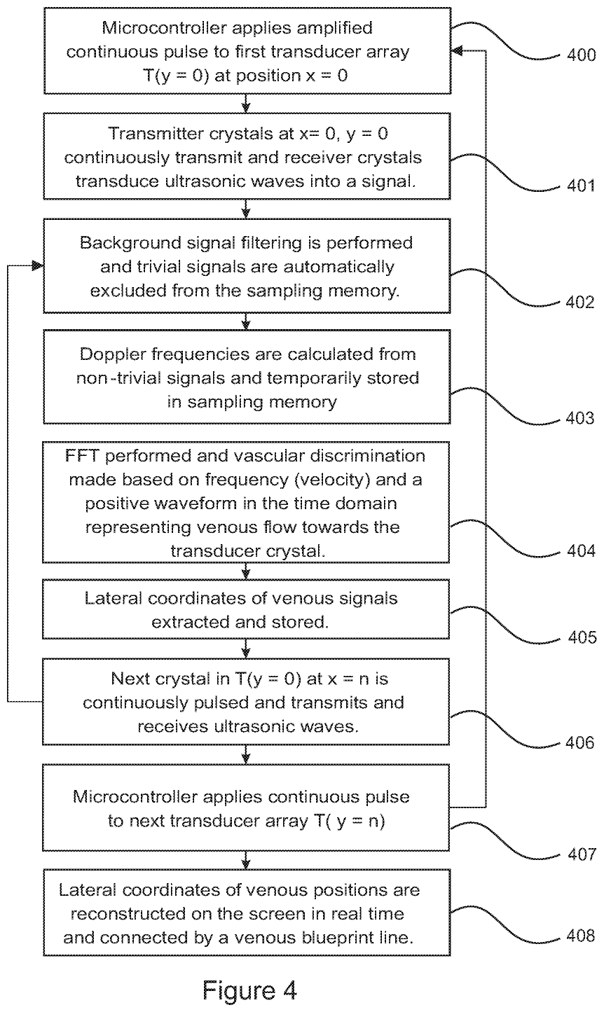

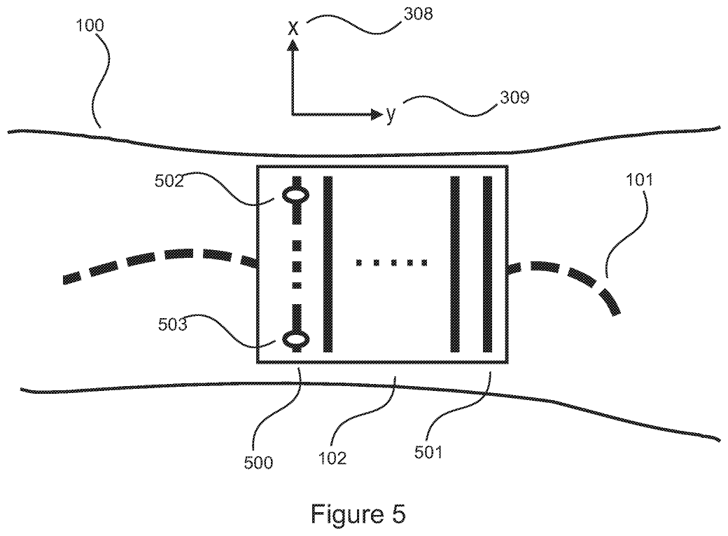

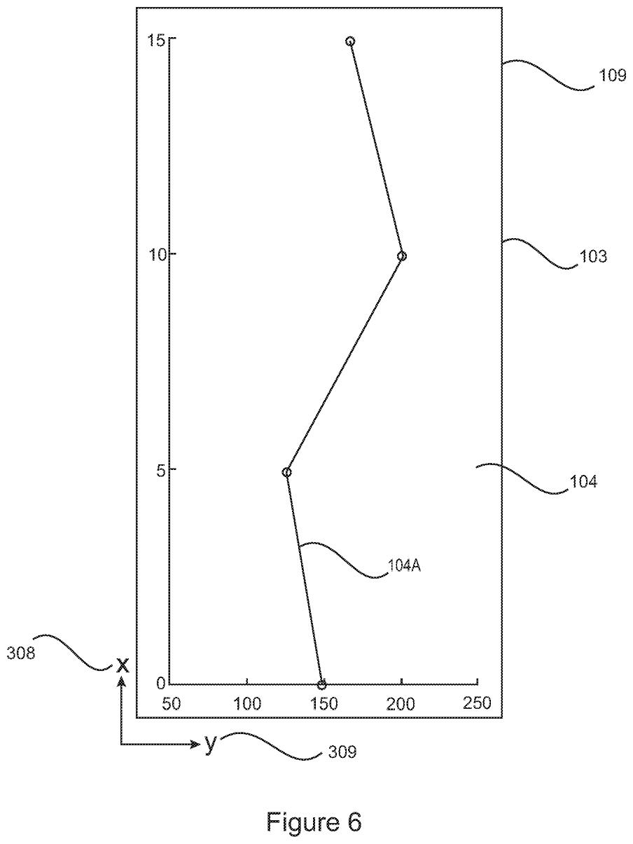

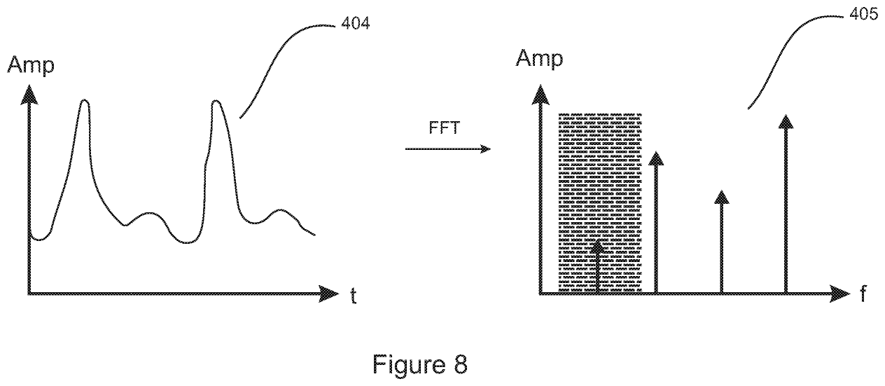

Turning now to FIG. 2, FIG. 3, FIG. 4, FIG. 5, FIG. 6 and FIG. 8, in summary, the portable ultrasound device 109 is—through selection by the controller 250 of transmission signals and processing of received echo signals by processor 350 and its associated processor blocks 208-210—able to locate peripheral veins 101 in the lateral direction 308, 309 (as shown in FIG. 3b) and at various selected depths from 5 mm to 2 to 3 cm, up to 7 to 15 centimetres in exceptional central vessel cases, and through dedicated processors perform signal processing 203, 205, 206 and digital processing block 208 to display a simplified longitudinal representation 104A of the peripheral vein 101 which can then be shown in real-time on the screen 104 of an integrated display unit 103, which updates and displays the vein 101 path as the portable ultrasound device 109 is moved over the surface of the skin of the patient's forearm 100.

Portable ultrasound device 109 allows discrimination between peripheral veins and arteries. In one embodiment, digital processing block 208 involves execution of an algorithm as embodied in electronic instructions allowing such discrimination through measurement of pulsatility. Veins are typically far less pulsatile than arteries, if pulsatile at all. Pulsatility metrics suitable for measurement and input to such an algorithm here include pulsatility index as described above as determined from velocity waveforms (if determined in the time domain) or high amplitudes at low frequencies (if determined in the frequency domain following Fast Fourier Transform (FFT) as schematically shown in FIG. 8). Digital processing block 208 may, in embodiments, then prevent any representation of an artery appearing on the screen 104 of integrated display unit 103.

FIG. 2 represents the system block diagram for the portable ultrasound device 109. Device 109 is provided with functionality for controlling and processing transmission and receival of ultrasonic waves from transducer crystal array 102 and the display or storage of imaged data. Excessive circuitry required for whole body imaging and not specifically necessary for vascular imaging are deliberately excluded from the system to reduce size and processing requirements and to ease use of the portable ultrasound device 109.

FIG. 2 illustrates a Field Programmable Gated Array 205, within controller block 250 which acts as a microcontroller for portable ultrasound device 109 and stores and generates pre-programmed digital signals containing ultrasonic oscillation settings, which is in turn converted to an analogue signal using a Digital to Analogue Converter (DAC) 204 and filtered for noise 203. In this embodiment, the filtered signal is sent to an ultrasonic waveform oscillator 201 which generates and transmits a primed signal to a diplexer 200 for frequency-domain multiplexing. It will be understood that FPGA 205 can be replaced with a chip-based controller, such as an ASIC chip based controller and there may be other acceptable alternatives.

In this embodiment, the ultrasonic waveform oscillator 201 can be powered by a high-voltage power source 202 which can be toggled on or off using the user interface 105. The array of transducer elements 102 thus transmits and receives ultrasonic signals at the pre-determined frequencies, say in the range 3 to 8 MHz with 7 MHz being selected here (this comparing with the wide frequency range 2 to 18 MHz for typical diagnostic sonographic scanners—see “Application of Ultrasound in Medicine” at www.ncbi.nlm.nih.gov>pmc>articles>PMC3564184—towards and from the examinee, respectively.

Received signals from transducer crystal array 102 are amplified using a low noise amplifier 207, filtered 203 and returned to the FPGA microcontroller 205 as a digital signal using an Analogue Digital Converter (ADC) 206. The received signals are processed onboard the portable ultrasound device 109 by processor block 210 using digital instructions or algorithms described below. As the calculations are complex, parallel processing is implemented at processor block 210.

Though on-board processing by processor 350 is most preferred, it is possible to reduce processing constraints on the portable ultrasound device 109 by performing processing using a cloud-based system such as that provided by Amazon AWS, whereby data are wirelessly transmitted, processed and returned to the device 109. In this example, trivial and junk signals are automatically excluded while Doppler frequencies are calculated for non-trivial signals and stored on the temporary sampling memory 209.

The digital processing block 208 executes instructions, by methods such as those described below, convert the raw signals into a simplified vein representation 104A which is displayed on screen 104 of the integrated display unit 103.

The display mode switch 106 is toggled to switch between a simplified vein representation 104A or conventional B-mode or colour-doppler images.

Data related to the ultrasound imaging process for portable ultrasound device 109 are stored on a device memory 211 or transferred to another device 212 by means of reversible USB-C, Bluetooth 5.0, or wireless internet in the form of standard WiFi, dual-band, Wi-Fi Direct or hotspot.

FIGS. 3a and 3b show an ultrasonic transducer crystal array 102 located at the base 102A of the housing 110 of portable ultrasound device 109 for transmitting and sensing reflected waves of the pulse ultrasonic signals. In this case, the base 102A is comprised of individual transducer elements 300 padded with a backing layer 302 and a matching layer 301. The transducer elements 300 are such that there is at least one emitter or transmitter crystal 705 and one receiver crystal 707 per transducer element 300 separated by a septum 706 with acoustic insulation properties though other arrangements of transmitter crystal 705 and receiver crystal 707 are possible. Transducer elements 300 may include a piezoelectric material such as PZT, or films, PVDF, PMN-PT, PMN-XX, PIN-PMT-XX where the XX represents the several derivatives of the materials, silicon, metal, CMUT, MEMS, NEMS and so on. The transducer elements 300 are conveniently selected from the group consisting of capacitative micromachined ultrasonic transducers (CMUT), piezoelectric micromachined ultrasonic transducers (PMUT) and similar configurations that use semiconductor technologies to produce the transducer elements 300 of the entire transducer crystal array 102.

Transducer elements 300 are connected to the electronics package 303 also including the on-board battery. In this example, the electronics package 303 is surrounded by a heat exchange and dissipation mechanism 305 which is in turn surrounded by an acoustic insulation layer 304.

The portable ultrasound device 109 includes a plurality of linear transducer arrays 300A, here four arrays, with the transducer arrays 300A—as shown in FIG. 3b—being oriented parallel to each other and carried by the housing 110 of device 109. The plurality of parallel transducer arrays 300A provides the ability to image vein 101 in multiple transverse planes to display the lateral position of the vessel on screen 104.

The parallel transducer arrays 300A are angled at an angle of insonation ϕ where 10<ϕ<60, as shown in FIG. 3b, to ensure the Doppler effect is captured and not nullified. The angled configuration, being fixed, also saves the user from requiring to manually adjust the operating field of view and imaging angles. In this example, parallel transducer arrays 300A are separated by a distance ξ along the horizontal axis 309, chosen to minimise interference and maximise the scanning window 708. ξ may, as described above, be in the range of 5-30 mm. In this embodiment, a 15 mm spacing is provided between each parallel array 300. This would cover a region of interest 45 mm. A spacing above 30 mm would make the portable ultrasound device 109 larger than desirable for use in a cannulation procedure.

FIG. 4 shows a flow diagram for a mode of ultrasonic signal acquisition for use in imaging the vein 101 for cannulation. The FPGA microcontroller 205 applies a continuous pulse throughout operation, thus providing continuous wave operation, starting with emitter crystals 705 of the first transducer array (T(y=0) at position x=0) at the boundary (step 400), shown in FIG. 5 at 503 of the first transducer 500. Reflected ultrasound waves received by receiver crystals 707 are transduced into a voltammetric signal (step 401) which is then filtered (step 402) to remove trivial signals from entering sampling memory 403.

Doppler frequencies and corresponding coordinates are temporarily stored in the sampling memory (step 403) before a Fast Fourier Transform (FFT) is applied to determine the received frequency and then calculate a frequency shift (Doppler shift). This Doppler shift can help discriminate veins 101 and arteries 101A based on an energy signal determined from the frequency shift, with positive waveforms 505 representing flow towards the transducer receiving crystals (i.e. blood flow in a vein or vice versa) 707 (step 404). This characterisation may be termed the venous signature of vein 101 which is stored and/or displayed (step 405) as venous path 104A on screen 104.

The Doppler shift can also be used to calculate blood flow rates or velocities according to the following formulae:

v=(frequency shift*speed of sound)/(2*transducer frequency*(cosine(angle of insonation ϕ))

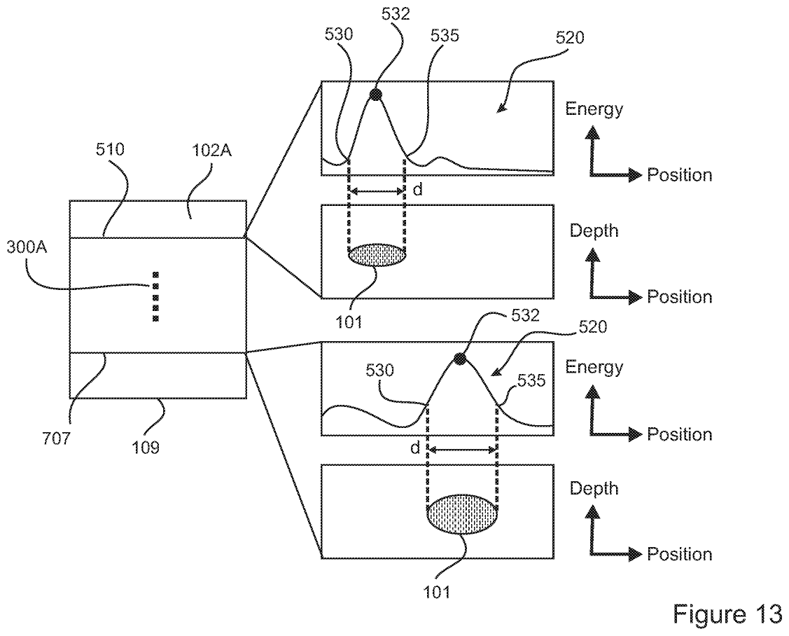

Flowrate=velocity*cross sectional area where cross sectional area is 0.25*pi*diameter² where diameter may, for example, be calculated as described below with reference to FIG. 13.

The next transducer crystals 705 are pulsed and the process from steps 402-406 is repeated until the nth crystal on the first transducer 502, as shown in FIG. 5, is reached. Once the first transducer 500 is cycled, an inter-transducer cycle begins until the nth transducer 501 is reached (step 407). The lateral coordinates x, y of each venous signature 104A per transducer element 300, 501, 502 are reconstructed laterally on the screen 104 in real time and connected by a venous blueprint line to form the image 104A of vein 101 as shown in FIG. 6 (step 408). This image 104A may be updated continuously on readily viewable screen 104 as the portable ultrasound device 109 is operated by the user.

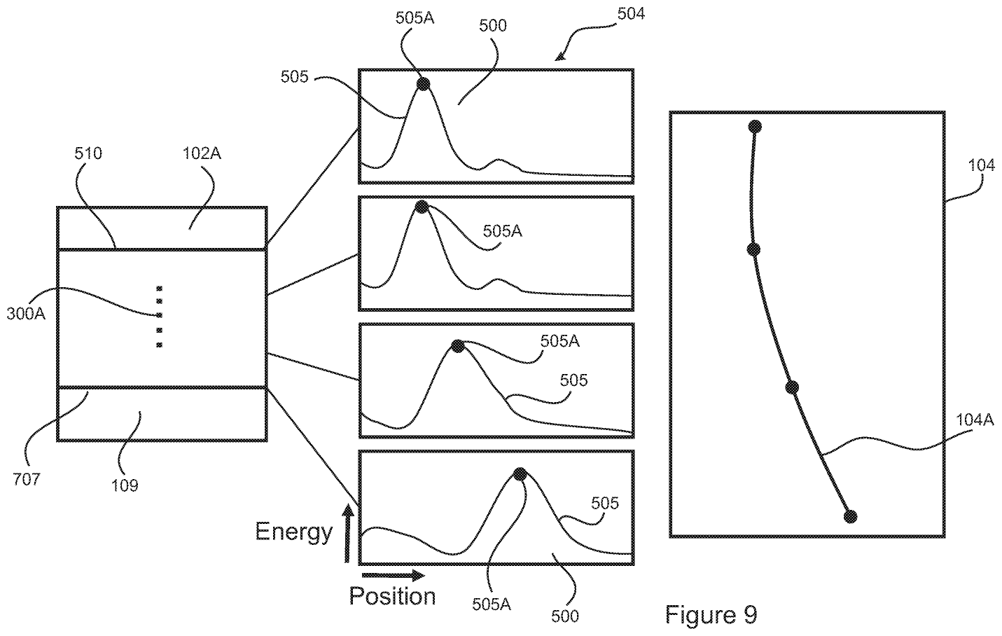

With further reference to step 404, the processor blocks 208-210 can alternatively be programmed with instructions to discriminate between arterial and venous structures using an energy signature 504 to determine the position of vein 101 below the contacting area of the ultrasound transducers 705, 707 with the patient's skin. The energy signature 504, as schematically indicated in FIG. 9, may be determined from a frequency-domain representation 405 of the raw signal, determined by a Fast Fourier Transform (FFT) of the continuously acquired current signal 404, as indicated by FIG. 8, or more preferably from a power spectral density (PSD) computed from the sampled signal. This energy signature is preferably the amplitude (Amp) of the dominant signal frequency, or alternatively the sum of amplitudes of dominant frequencies or else the sum of amplitudes squared. More preferably the energy is the area under the FFT or PSD distribution. Alternatively, the energy could be the ratio of amplitude to area under the distribution or the ratio of sum of amplitudes (or sum of amplitudes squared) to the area under the distribution.

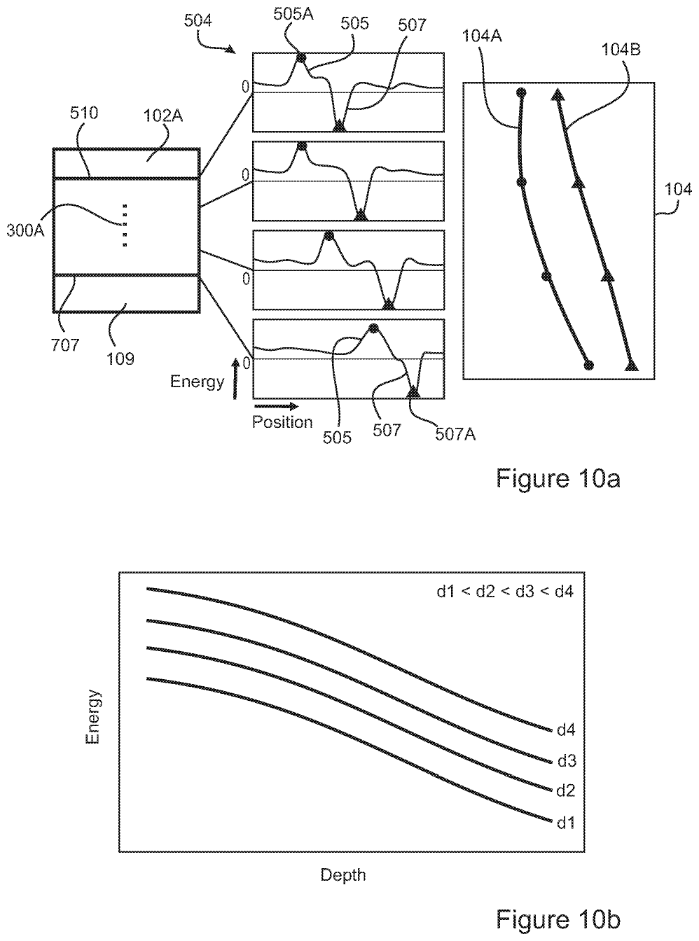

Further referring to FIG. 9, and as above described, the positive waveform 505 obtained for each of receiver crystals 510 and 707 and receiver crystals within transducer element array 300A is representative of a point along a vein 101, these points having a position which can stay the same or vary as shown in the energy-position trace or waveforms 504 and, as shown in FIG. 10 for artery 101A, waveform 507. Point 505A or the peak of the waveform 505 approximately represents the centre of the vein 101 at a given point with maximum energy typically being at the centre of a healthy vascular structure. A negative waveform 507, as shown in FIG. 10, would be representative of a point along an artery 101A with point 507A or the peak of the waveform 507 approximately representing the centre of the artery 101A at a given point with maximum energy typically being at the centre of a healthy vascular structure. The processor 350 displays the points on screen 104 with the result showing the path of vein 101 as line 104A.

The processor 350 may allow discrimination between a vein 101 and an artery 101A using the difference between positive (vein 101) and negative (artery 101A) waveforms 505, 507. Vein path is shown as line 104A and artery path is shown as line 1046.

The processor 350 also estimate depth of vein 101 below the skin by executing the above described energy signature based instructions and as also schematically indicated by FIG. 10. For a vessel diameter, d1-d4, calculated using methods described below, characteristic curves may be inferred relating magnitude of energy to the vascular structure or vein 101 depth for different vessel diameters d1 to d4 with d1<d2<d3<d4 as shown in FIG. 10(b) with larger vessels having a higher magnitude energy signature. This depth can be determined, and displayed on screen 104, as a value in millimetres or centimetres below the skin, or through colour weightings by depth magnitude or through other visual representations such as 3D perception of shallowness or depth. The processor 350 thus allows calculation of depth of vein 101 or artery 101A beneath the skin without the need for B-mode cross sectional image reconstructions.

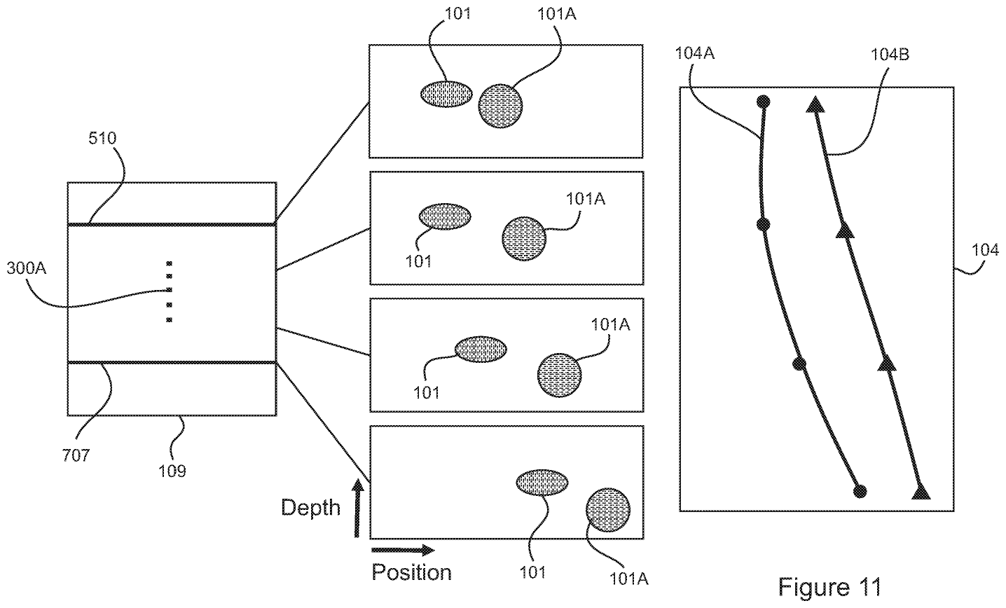

Alternatively to the approaches described above, B-mode imaging may be performed for each transducer 510, 707 within the array as schematically shown in FIG. 11. Structural metrics (such as diameter, compressibility (as indicated), and other curve features) can be calculated by processor 350 and used to discriminate between arteries 101A and veins 101 with paths of vein 101 and artery 101A being displayed as respective lines 104A, 104B on screen 104. To this end, machine learning techniques, for example including the use of convolutional neural networks, can be used to classify and identify veins and arteries, following training datasets produced by a population of experienced ultrasound users. In B-mode or colour Doppler representations, depth of vascular structures can be directly measured and displayed on screen 104 of ultrasound device 109.

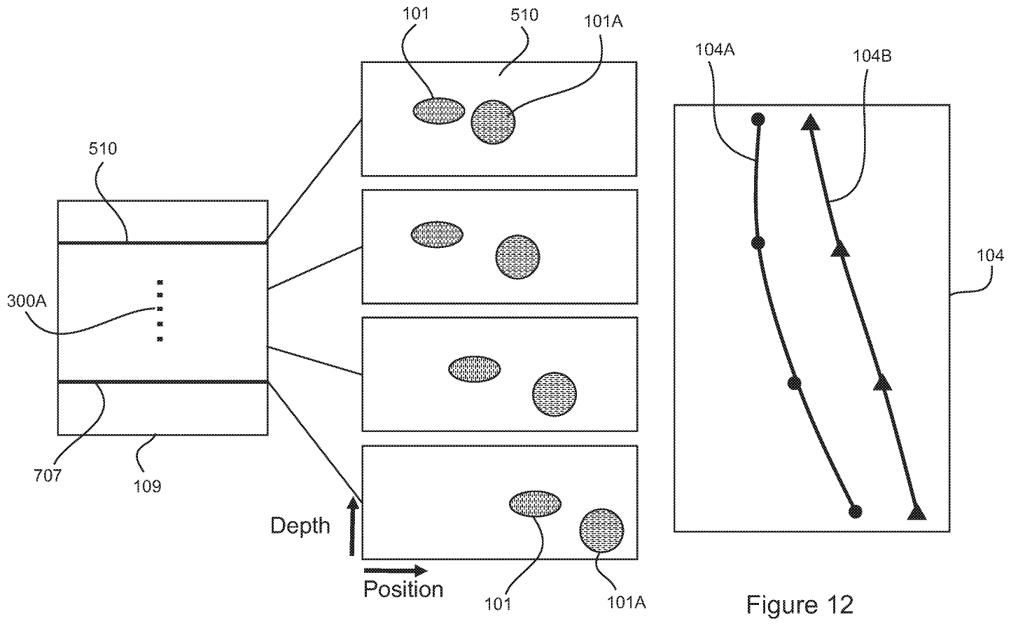

In another embodiment of an algorithm for artery or vein discrimination, as schematically shown in FIG. 12, B-mode and Colour Doppler images 510 are acquired and automatic computer vision techniques are used to identify flow away or towards the transducer 510, 707 (or flow away from the heart, representing artery 101A, and flow towards the heart, representing vein 101). Colour Doppler represents flow towards the transducer (veins typically) as shades of blue 615 with intensities representing velocity magnitudes, and flow away from the transducer (arteries typically) as shades of red 520. As such, artery 101A and vein 101 can be easily distinguished through automatic computer vision techniques discriminating colour. In B-mode or colour Doppler representations, path and depth of vessels can be directly measured and displayed on screen 104 of ultrasound device 109 as vein path 104A and artery path 104B.

Conveniently, the processor 350 recognizes—in the energy signal based algorithms as described with reference to FIGS. 9, 10 and 13—that the peak in the energy signature forms part of parabolic or gaussian resembling distributions and that the start and end points 530, 535 of such distributions can be used to estimate vein 101 diameter, d, particularly in the lateral direction. A method of determining start or end point 530, 535—as indicated in FIG. 13—involves calculation, by processor 350, of gradient at every point along the energy curves 520 and computed by taking the ratio of energy change between consecutive data points to the change in position. A start point 530 may be identified, for example, when a gradient quickly increases from low values (say between −0.25 to 0.25) to a large value (say between 0.75 to 1). Conversely, an end point 535 may be identified as a change in gradient from a large negative value (say between −0.75 to −1) to a low value (say between −0.25 to 0.25). Compared to structural analysis of B-mode cross-sectional images which requires either manual measurements of diameter or automated feature recognition (through artificial intelligence constraints or machine learning methods) for diameter measurements, the energy curve method as described above allows for a more efficient and more objective method to determine diameter. Additionally, no training datasets (labelled) are needed for the automatic diameter calculation, however training machine learning models to optimize the identification of start and end points 530, 535 is possible.

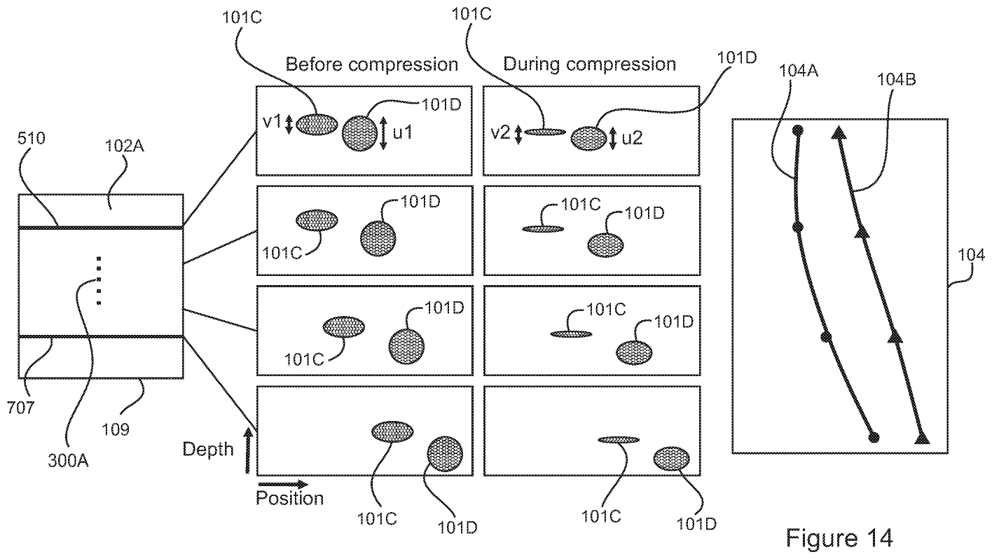

In another embodiment of the method of artery or vein discrimination, ultrasound data is acquired by ultrasound device 109 without and with mechanical compression of the subcutaneous vessels through application of external force by the housing onto the skin. This method is indicated in FIG. 14. Taking advantage of different vascular wall properties of veins (compressible but not muscular and not elastic) and arteries (muscular, less compressible than veins and elastic), changes during compression can be measured to distinguish between arteries and veins.

In one embodiment of the compression method, peak energy signatures (determined from either FFT or PSD) change with compression as schematically shown in FIG. 14. Notably, the positive peaks 101C (representing a flow in a vein 101 when flowing towards the receiver crystals 510, 707 and those in transducer array 300A) is flattened with compression while the negative peaks 101D (representing a flow in an artery 101A when flowing away from the transducer), only shows reductions in magnitude while maintaining its directionality (negative peak representing flow away from the transducer). The characteristic drop in the peak 101C (toward zero) for the vein and a linear decrease in magnitude for the artery 101D are preferably calculated through absolute value measurements or more preferably through feature curve analysis to discriminate between arteries and veins. Alternatively, as vascular structure diameter, d, can be determined from energy waveforms 504 as previously described, the change in vascular structure size due to compression can be estimated by calculating the change in diameter from before compression to after compression, i.e.—from u1 to u2 and from v1 to v2. A large deformation (say equivalent to 90-100% of the original diameter) is expected for veins compared to arteries. This method also results in vein path 104A and artery path 104B being displayed on screen 104 of ultrasound device 109.

In another embodiment of the compression method utilizing B-mode or colour doppler mode, automatic feature edge recognition is used to identify circular or elliptical objects and the maximum vertical chord length of said objects can be measured. Circular or elliptical objects can respectively be identified as arteries or veins as the reduction in maximum vertical chord length is largest for veins (say equivalent to 90-100% of the original diameter) compared to arteries.

In another embodiment of both compression methods described, the compressibility of the vein either through energy drops or chord length drops can be used to indicate the structural integrity of the vein 101 with larger resistance to compression indicating higher stiffness (structural integrity). Additionally, the restoration of structure following compression can be used to determine the extent of plastic deformations and therefore can be used to determine elasticity. The choice of structurally stable veins through both stiffness and elasticity inferences is a critical factor for cannula insertions as well as cannula success throughout in-dwelling periods (no dislodgement, extravasation).

Automatic feature recognition methods can be used in embodiments of B-mode or Doppler ultrasound, as described above, to automatically determine circular or elliptical objects within the field of view and vessel diameter or ellipticity can be calculated automatically by the processor. Based on vessel diameter or ellipticity, cannula size may be determined by the processor and displayed on the screen. For example, in adults, for vein diameters greater than 1.3 mm, the algorithm would most preferably recommend green cannulas (18G; 1.3 mm diameter), pink cannulas (20G; 1.1 mm diameter) or blue cannulas (22G; 0.9 mm diameter). Orange (14G; 2.1 mm diameter) or gray (16G; 1.8 mm diameter) may also be recommended for larger veins (above 1.8 mm), however these are recommended in trauma, resuscitation, rapid blood transfusions, rapid fluid replacement, or surgery, all at very high infusion flow rates (240 mL/min for orange and 180 mL/min for gray).

Calculated haemodynamic parameters such as shear stress or turbulence can be indicative of disruption of flow due to the inserted vascular access device, for example a cannula. Such parameters may be monitored to facilitate a cannulation process, for example—and without limitation—by determining a recommended time for replacement of the vascular access device and/or to assess the effectiveness of vein flushing with saline to keep the vein 101 open. For example, the processor 360 can calculate and represent blood flow rates on screen 104 and use this information to manage a cannulation. Procedure, for example, at a saline flow of 10 mL/h, blood stasis can be indicated on screen 104, which may occur around the cannula tip as a recirculation (stasis) zone is formed, hence blocking the tip and reducing device patency. Preferably, at 20 mL/h reduction in stasis is indicated and a score for vein patency may be calculated. Most preferably, 30-40 mL/h saline is indicated as being most effective for a larger range of venous flow rates and peripheral vein sizes. However, based on patient needs, determined by factors such as hydration, difficulty of access or bruising, larger flow loads above 40 mL/h can be indicated on screen 104 of ultrasound device 109.

Use of portable ultrasound device 109 is described below.

As the user moves the portable ultrasound device 109 over the skin of the patients forearm 100 to find a vein pathway (which will be determined, through use of the device 109, as vein 101), the user aims to align the simplified longitudinal representation 104A of the vein 101, obtained (preferably with other procedural data or recommendations as above described) with the notch on the base 102A.

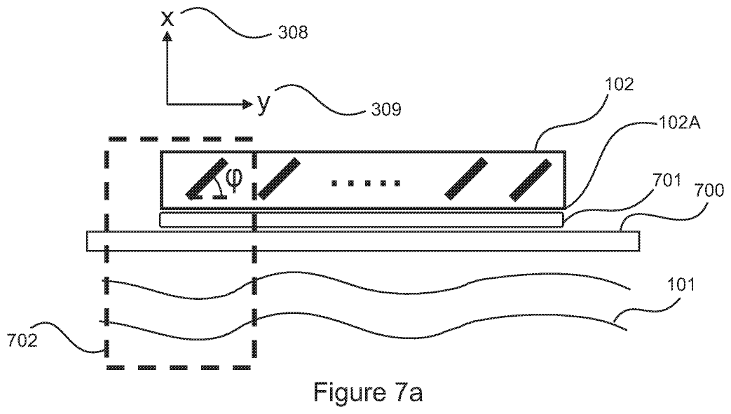

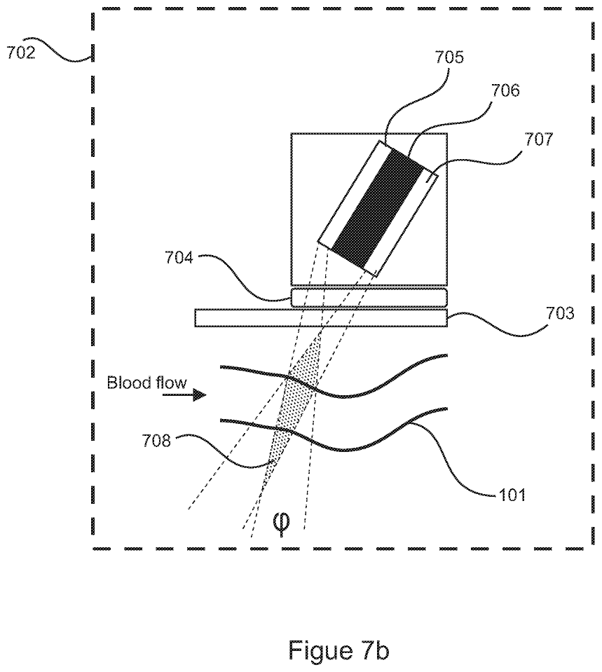

FIG. 7 shows an example where the user attaches a double-sided consumable acoustic conductive patch 701 to the base 102A of device 109, preferably through means of a medically compatible removable glue or a double-sided sticky tape. The sticky affixation end of the acoustic conductive patch 701 attaches to the base 102A of the portable ultrasound device 109. The opposite end of the acoustic conductive patch 701 can be peeled off to expose a medically sterile gel, contained within the patch 701, which aims to reduce conductance of ultrasonic waves through air, typically the cause of signal noise and signal loss, while maintaining the sterile field needed for cannulation.

Referring again to FIG. 1, once aligned, the user deploys a detachable fastening means 107 to secure the portable ultrasound device 109 in position on the patient's forearm 100. The fastening means 107 can be a single or multiple use strap or band or belt made of common medical grade material such as fabric or silicone, with an adjustable clasp to adjust for fastening force. The fastening force can be likened to that produced of a commonly used tourniquet, required to increase intra vascular pressure and hence engorge the vessel and improve visibility and reduce blood velocity to a range pre-programmed to be extracted by digital processing algorithms 208.

The user can then conveniently proceed to inserting a needle or cannula through an insertion notch 310 at the base 102A of the portable ultrasound device 109, without needing to hold it in place. Hence, the simplified longitudinal representation 104A of peripheral vein 101—produced by processor 350 and, in particular, processor blocks 208-210 as described above—guides the operator to the optimal spot of needle insertion with respect to the imaged pathway 104A of the peripheral vein 101 with benefits for both user and patient.

The portable ultrasound device 109 as described is self-contained, conveniently hand-held, low cost and simply structured. In contrast to conventional ultrasound equipment, the portable ultrasound device of the present invention does not require modular and separate system components consisting of scanning probes, processing units and monitors. The key components, including the transducer elements, processor and circuitry and screen are packaged in a single housing. The portable ultrasound device 109 is conveniently lightweight and may be used to assist venepuncture or cannulation and other ultrasonic imaging of sub cutaneous structures by a wider range of personnel without formal specialised training including medical and paramedical professionals such as, but not limited to, registered nurses, laboratory phlebotomists, therapists and researchers.

It will be understood that modifications and variations may be made to the portable ultrasound device and method of ultrasonic imaging of sub-cutaneous structures may be apparent to the skilled reader of this disclosure. Such modifications and variations are deemed within the scope of the present invention.

1. A portable ultrasound device for non-invasively imaging a selected sub-cutaneous structure in a subject, comprising:
(a) a housing;
(b) a plurality of arrays of transducer elements, each array being obliquely angled and arranged in parallel and each transducer element comprising a transmitter transducer and a receiver transducer, located within said housing for continuously transmitting ultrasound energy in a predetermined frequency range toward a body of a subject and continuously receiving echo signals in a predetermined frequency range from the body of the subject following reflection of ultrasound energy, said plurality of parallel arrays enabling imaging of a sub-cutaneous structure in multiple transverse and lateral planes;
(c) a controller for operating said plurality of arrays of transducer elements in a continuous wave doppler mode and communicable with a processor for processing said echo signals from said plurality of arrays of transducer elements; and
(d) a screen forming part of said housing for displaying an image of said sub-cutaneous structure wherein said processor is configured to process said echo signals returning from the sub-cutaneous structure to selectively produce an interpretable image of the sub-cutaneous structure of the subject.

2. The ultrasound device of claim 1, wherein each transducer element comprises a transmitter transducer interleaved with a receiver transducer.

3. The ultrasound device of claim 1, wherein said sub-cutaneous structure is a vascular structure.

4. The ultrasound device of claim 1, wherein said plurality of arrays of transducer elements are spaced apart from each other by a distance ξ along a horizontal axis selected to minimise interference and maximise a scanning window.

5. The ultrasound device of claim 4, wherein ξ is between 5 and 30 mm.

6. The ultrasound device of claim 3, wherein said parallel arrays are angled at an angle of insonation Φ where 10 degrees<Φ<60 degrees.

7. The ultrasound device of claim 6, wherein said screen, with the assistance of the processor, provides an indication of the correct location for insertion of a cannula or like device into the sub-cutaneous structure and representation(s) on the screen optionally displaying information including one or more of: a depth of an imaged sub-cutaneous structure; and a position of a needle tip being inserted into the sub-cutaneous structure.

8. The ultrasound device of claim 7, wherein said processor is programmed to calculate an optimal needle gauge and/or insertion angle recommended for access to the imaged sub-cutaneous structure.

9. The ultrasound device of claim 7, wherein the representations are provided in 3D for both a vascular structure and haemodynamic fields, optionally including one or more of: velocity, pressure, shear stress, turbulence, stagnation, pulsatility or stenosis.

10. The ultrasound device of claim 1, wherein said processor is programmed with instructions to discriminate between arterial and venous vascular sub-cutaneous structures.

11. The ultrasound device of claim 10, wherein said processor discriminates between the arterial and venous sub-cutaneous structures based on measurement of pulsatility.

12. The ultrasound device of claim 10, wherein said processor is programmed with instructions to discriminate between the arterial and venous sub-cutaneous structures based on processing of an energy signal determined from a Fast Fourier Transform (FFT) of a sampled ultrasound signal.

13. The ultrasound device of claim 10, wherein said processor discriminates between the arterial and venous sub-cutaneous structures based on a power spectral density (PSD) computed from a sampled ultrasound signal.

14. The ultrasound device of claim 13, wherein said processor is programmed with instructions to determine the position of the sub-cutaneous structure below a contacting area of the ultrasound device within the body of the subject based on the processing of the sampled ultrasound signal.

15. The ultrasound device of claim 14, wherein the processor determines at least one of a depth and a dimension of the sub-cutaneous structure below said contacting area based on processing of the sampled ultrasound signal.

16. The ultrasound device of claim 14, wherein the processor processes the sampled ultrasound signal with compression of the sub-cutaneous structure.

17. A method for imaging a sub cutaneous structure in a subject, comprising:
non-invasively and continuously transmitting ultrasound energy in a predetermined frequency range to the body of the subject in continuous wave doppler mode via a plurality of arrays of transducer elements contained in a portable ultrasound device applied at or proximate to a location on the body of the subject, each array of said plurality of arrays of transducer elements being obliquely angled and arranged in parallel and each transducer element comprising a transmitter transducer and a receiver transducer, said plurality of parallel arrays of transducer elements enabling imaging of a sub-cutaneous structure in multiple transverse and lateral planes;
receiving echo signals in a predetermined frequency range from the body of the subject following transmission of ultrasound energy;
processing said received echo signals with a processor; and
producing an image displaying the sub-cutaneous structure of the subject on a screen forming part of the portable ultrasound device.

18. The imaging method of claim 17, wherein each transducer element comprises a transmitter transducer interleaved with a receiver transducer.

19. The imaging method of claim 17, wherein said processor discriminates between arterial and venous vascular sub-cutaneous structures.

20. The imaging method of claim 19, wherein said processor discriminates between arterial and venous sub-cutaneous structures based on measurement of pulsatility.

21. The imaging method of claim 17, wherein said processor discriminates between arterial and venous sub-cutaneous structures based on processing of an energy signal determined from a Fast Fourier Transform (FFT) of a sampled ultrasound signal.

22. The imaging method of claim 17, wherein said processor discriminates between arterial and venous sub-cutaneous structures based on a power spectral density (PSD) computed from a sampled ultrasound signal.

23. The imaging method of claim 17, wherein said processor determines a position of the sub-cutaneous structure below a contacting area of the ultrasound device within the body of the subject based on the processing of the sampled ultrasound signal.

24. The imaging method of claim 23, wherein the processor determines at least one of a depth and a dimension of the sub-cutaneous structure below said contacting area based on processing of the sampled ultrasound signal.

25. The imaging method of claim 23, wherein the processor processes the sampled ultrasound signal with compression of the sub-cutaneous structure.

26. The imaging method of claim 17, wherein the processor calculates an optimal needle gauge and/or insertion angle for access to an imaged sub-cutaneous structure, for example a vascular structure, by a vascular access device.

27. A non-transitory computer-readable medium carrying one or more sequences of instructions, wherein execution of the one or more sequences of instructions by one or more processors causes the one or more processors to perform the steps of:
transmit a signal to a plurality of arrays of transducer elements in portable ultrasound device to cause the plurality of arrays of transducer elements to non-invasively and continuously transmit ultrasound energy in a predetermined frequency range to a body of a subject in continuous wave doppler mode applied at or proximate to a location on the body of the subject, each array of said plurality of arrays of transducer elements being obliquely angled and arranged in parallel and each transducer element comprising a transmitter transducer and a receiver transducer, said plurality of parallel arrays of transducer elements enabling imaging of a sub-cutaneous structure in multiple transverse and lateral planes;
receive echo signals in a predetermined frequency range from the body of the subject following transmission of ultrasound energy;
process said received echo signals to determine image data of the sub-cutaneous structure of the subject; and
transmit a signal to a screen forming part of the portable ultrasound device to produce an image on the screen displaying the sub-cutaneous structure of the subject based on the image data.
